(19) United States
(12) Patent Application Publication
(10) Pub. No.: US 2025/0369187 A1
(43) Pub. Date: Dec. 4, 2025

(54) METHOD AND DEVICE FOR PRODUCING A MICROFIBRILLATED CELLULOSE FILM

(71) Applicant: Stora Enso OYJ, Helsinki (FI)

(72) Inventors: Otto Nylén, Helsinki (FI); Kaj Backfolk, Lappeenranta (FI); Anni Karppinen, Espoo (FI)

(73) Assignee: Stora Enso OYJ, Helsinki (FI)

(21) Appl. No.: 18/872,947

(22) PCT Filed: Jun. 9, 2023
(86) PCT No.: PCT/IB2023/055967

§ 371 Date: Dec. 9, 2024

(30) Foreign Application Priority Data
Jun. 10, 2022 (SE) 2230187-3

(51) Int. Cl. D21H 11/18; D21G 9/00

(52) U.S. Cl. CPC D21H 11/18; D21G 9/0054

(57) ABSTRACT

A method of producing a microfibrillated cellulose. MFC. film from an MFC dispersion, comprises subjecting a wet MFC film (F) on a support to at least one drying step to form a dry MFC film (F′). measuring at least one parameter indicative of a concentration of at least one component, such as an additive or an impurity, and performing at least one of the tasks: i) adjusting at least one production parameter which influences the concentration of said at least one component in response to said at least one parameter. and ii) recording said at least one parameter for each of said data points to provide a chemical map of the dry MFC film (F′). comprising a plurality of data points which are each associated with a respective position on the dry MFC film (F′).

### TECHNICAL FIELD

The present disclosure relates to a method and a device for producing microfibrillated cellulose, MFC, films. The disclosure relates particularly to a method which may provide a high quality MFC film, in particular wherein variations in the content of additives may be minimized and/or wherein impurities may be at least identified and managed. The disclosure further relates to devices for producing such MFC films and to the use of such devices for producing an MFC film. The disclosure also relates to an MFC film, which is produced according to the method.

The method and device find particular application in continuous production of MFC film.

### BACKGROUND

Microfibrillated cellulose (“MFC”) shall in the context of the patent application mean a cellulose particle, fiber or fibril having a width or diameter of from 20 nm to 1000 nm.

Various methods exist to make MFC, such as single or multiple pass refining, pre-hydrolysis followed by refining or high shear disintegration or liberation of fibrils. One or several pre-treatment steps is usually required in order to make MFC manufacturing both energy efficient and sustainable. The cellulose fibers of the pulp used when producing MFC may thus be native or pre-treated enzymatically or chemically, for example to reduce the quantity of hemicellulose or lignin. The cellulose fibers may be chemically modified before fibrillation, wherein the cellulose molecules contain functional groups other (or more) than found in the original cellulose. Such groups include, among others, carboxymethyl (CM), aldehyde and/or carboxyl groups (cellulose obtained by N-oxyl mediated oxidation, for example “TEMPO”), or quaternary ammonium (cationic cellulose). After being modified or oxidized in one of the above-described methods, it is easier to disintegrate the fibers into MFC.

MFC can be produced from wood cellulose fibers, both from hardwood and/or softwood fibers. It can also be made from microbial sources, agricultural fibers such as wheat straw pulp, bamboo, bagasse, or other non-wood fiber sources. It can be made from pulp, including pulp from virgin fiber, e.g. mechanical, chemical and/or thermomechanical pulps. It can also be made from broke or recycled paper or similar packaging substrates.

Current research indicates that MFC may be a suitable material for packaging and coating of packaging, due to its barrier properties. Hence, MFC has the potential of replacing or supplementing currently used barrier films and layers, including polymer and metal films and coatings.

Forming of MFC films can be achieved by solvent casting of a viscous or gel-like fluid material on a support, such as a continuous conveyor belt, followed by dewatering/drying (e.g. evaporation) of the solvent.

The term “solvent casting” is a known term designating methods wherein a film is produced by applying a wet film comprising a film forming component which is distributed in a medium that is to be essentially removed, for example by dewatering and/or evaporation. The film forming component may be dispersed in a dispersing medium or dissolved in a solvent, hence the term “solvent casting”.

In the following, the term “MFC dispersion” will be used as reference to a dispersion/suspension or solution containing MFC and a dispersing medium, which is frequently water. The MFC dispersion will be in a viscous state.

Utilization of MFC films in packaging applications involves a specific challenge due to brittleness of the MFC film. Elasticity and ductility of the MFC film are associated with the water present in the MFC film, and the brittleness occurs when the MFC film is too dry. The dryness and subsequent brittleness can be a local effect in the MFC film, especially at the edge area of the film. In a casting process the lateral edges of the MFC film tend to dry faster than the middle parts of the film, which results in cross-machine (CD) directional differences in water content and ductility and subsequently brittle edges. In addition, uneven drying can lead to inhomogeneous distribution of additives in the film. Due to brittleness, especially at the edge areas, MFC films tend to break easily in converting processes. The problem is not necessarily limited to narrow and low speed appearing in lab and pilot-scale fabrication processes and use of films, but becomes more critical when operating film manufacturing on wider machines and/or in bigger scale and in higher production speeds.

A known approach is to use plasticizers and/or humectants, such as sugar alcohols (e.g. sorbitol) or polyethylene glycol, to make MFC films more ductile. Such hygroscopic additives and the water that they bind to the film may, however, interfere with bonding of the microfibrils, increase the strain-at-break value and decrease the tensile strength. Use of such additives improves the overall level of film ductility, but sometimes the films are still too brittle and the problem with cross-machine directional variation of water content of the film, and brittle edges, also remain.

An additional problem relates to manufacturing of MFC films with casting technology when impingement, infra-red (IR), or any other thermal drying from top of the film is used to dry films on a non-porous casting support. The casting support needs to be slightly wider in cross-machine direction than the wet MFC film deposited on it. However, since the heat flux applied from above increases the temperature of the casting support edges which have no wet MFC layer on them, the lateral edge portions of the film will be subjected to additional heat. This additional heat at the casting support edges makes the MFC film dry faster at the edges. MFC films are very thin and get easily overdried.

Moreover, when MFC films are allowed to have an uneven moisture or additive profile in CD, the release behavior or adhesion of dry or semi wet MFC films to the casting support changes and becomes unpredictable or has higher variations. Since the film dries faster at the edges than at the middle, the edges tend to lose the adhesion to the casting support earlier. This also results in deformed and damaged edges in the film and web breaks in MFC film manufacturing. In the production of MFC film, one or more types of property-modifying additives may be added to the MFC dispersion. Non-limiting examples of such additives include softening agents, film forming agents, and additives promoting barrier properties and/or stretch properties.

Further, in the production of MFC film, there is a desire to increase dry solids content in the device from which the MFC dispersion is applied to the support, as this facilitates the drying process, in that less dispersing medium needs to be removed.

However, the increase in dry solids content makes the MFC dispersion more viscous and thus increases the risk of additives being unevenly distributed in the MFC dispersion. On the other hand, mixing the MFC dispersion too much may lead easily to aggregation of fibers and/or consumes energy. Therefore, it is important to control the mixing according to the process data.

Increased temperature can be used for reducing the viscosity of the MFC dispersion and thus making the mixing easier. In addition, controlling the pH or conductivity of the MFC dispersion might be needed to achieve an even distribution of any additives.

A challenge in the subsequent drying of the MFC dispersion is that the dewatering and drying process causes in-plane and out-of-plane movement of dispersing medium within the wet film, as well as migration of additives, and in particular additives which are soluble in the dispersing medium and/or which are not prone to adhere to the MFC fibers, in the film after it has been applied onto the support.

Such migration may have an impact on various film properties, such as runnability, barrier properties and/or mechanical performance.

Hence, there is a need for improvements in the conversion of an MFC dispersion to an MFC film on a support. There is also a need for further improvement in MFC film properties, and in particular with regard to the homogeneity of such MFC film properties throughout the MFC film.

### SUMMARY

It is an object to provide a method and a system, which provide improved MFC film quality, preferably with limited or no increase in production cost and more preferably with a reduction in production cost. A particular object is to address problems associated with inhomogeneous film properties, such as barrier properties, local brittleness, and/or problems with runnability in film manufacturing and converting.

The invention is defined by the appended independent claims, with embodiments being set forth in the appended dependent claims, in the following description and in the attached drawings.

According to a first aspect, there is provided a method of producing a microfibrillated cellulose, MFC, film from an MFC dispersion. The method comprises providing an MFC dispersion comprising a dispersing medium and a film forming component comprising about 50-100% by weight MFC, applying a layer of the MFC dispersion to a support to form a wet MFC film, subjecting the wet MFC film on the support to at least one drying step to form a dry MFC film, measuring at least one parameter indicative of a concentration of at least one component, such as an additive or an impurity, in the wet MFC film and/or in the dry MFC film at at least two data points which are laterally spaced across a width of the MFC film, and/or which are longitudinally spaced along a length direction of the MFC film. The method further comprises performing at least one of the tasks:
i) adjusting at least one production parameter which influences the concentration of said at least one component in the MFC film in response to said at least one parameter, and
ii) recording said at least one parameter for each of said data points to provide a chemical map of the dry MFC film, comprising a plurality of data points which are each associated with a respective position on the dry MFC film.

A content of the dispersing medium of the MFC dispersion may be at least 7530% by weight, preferably more than 80% by weight, more than 85% by weight, more than 90% by weight or more than 95% by weight. The film forming component may comprise, consist of or consist essentially of MFC, optionally with one or more water soluble polymers which may operate as co-additives and/or co-film formers. Thus, the MFC dispersion comprises a dispersing medium and a film forming component, wherein the film forming component comprises 50-100% by weight MFC (i.e. based on total dry weight of the film forming component). For example, the film forming component may comprise, in addition to MFC, a water soluble polymer that can form a film and/or improve bonding between the cellulose fibrils. Typical examples of such polymers are e.g. natural gums or polysaccharides or derivatives thereof such as e.g. carboxymethylated cellulose (CMC), starch, or PVOH or analogues thereof. The film forming component may also comprise one or more further additives, such as one or more property-modifying additives. Non-limiting examples of such additives/chemicals are softeners and plasticizers, such as glycols, sugar alcohols such as sorbitol or polysaccharides such as sorbitol or glucose, film forming agents such as polyvinyl alcohol (PVOH), carboxymethylated cellulose or methylcellulose, fillers, pigments, retention chemicals and dispersants or other polyelectrolytes, latexes, cross-linkers, optical dyes, fluorescent whitening agents, de-foaming chemicals, salts, pH adjustment chemicals, surfactants, biocides and/or optical chemicals. The film forming component may also comprise other natural fibre material in addition to MFC.

The layer of the MFC dispersion may be applied to the substrate by a casting technique.

The support may be a non-porous support and in particular a continuous non-porous support, such as a metal belt, in particular a steel belt, a polymer belt or a polymer coated belt. A metal belt may be coated, e.g., with ceramic material.

The dispersion medium may comprise water and optionally one or more solvents.

By measuring a parameter indicative of a concentration of at least one component, such as an additive or an impurity, which is present in the MFC dispersion at at least two data points which are spaced apart in at least one of a width direction and a length direction of the MFC film, it is possible to identify a variation in component content across the width and/or along the length of the film.

For example, the component (which also may be denoted as e.g. constituent) may be a compound, an atom or an ion.

The component may be an additive that has been intentionally added in order to modify a property of the wet or dry film. Alternatively, the component may be an impurity, which may originate from any place upstream of the point where the measurement is made.

The parameter that is measured may, as a non-limiting example, be a spectroscopic response, which may directly indicate the presence and concentration of the component. As yet another example, the parameter may be e.g. a temperature, which may indirectly indicate the presence or absence of a component that correlates with e.g. dispersing medium content.

Such variations may only become visible after the MFC film has been dewatered and/or at least partially dried.

The measuring may be performed substantially continuously during the production of the MFC film.

Based on an identification of such variation, it is possible to take appropriate action to adjust at least one production parameter in order to ensure an even distribution of the component in the MFC film.

The production parameter may, as non-limiting examples, be a drying condition for the MFC film, a dewatering condition for the MFC film, a mixing condition for the MFC dispersion or an application condition for the MFC dispersion. The production parameter may also be the amount of MFC, dispersing medium and/or component(s) provided in the MFC dispersion, as well as the point and/or time in the process where they are being added.

It is also possible to generate a chemical map of the MFC film. Such map may be provided as a 2D map of the dry MFC film, where only 2D data is measured, or as a 3D map where data is also collected in the thickness direction of the MFC film.

A 2D map would provide data on the MFC film in a plane formed of the width direction (cross machine direction) and the length direction (machine direction or length direction) of the MFC film. A 3D map would also comprise data in a thickness direction (z direction) of the MFC film.

The map may show how the concentration of one or more components varies throughout the MFC film. The map can be utilized in the downstream handling of the film. For example, areas or parts of the MFC film, as seen in the length direction with too high content of the component(s) can be removed from the dry MFC film, e.g. from a roll of the dry MFC film according to the 2D map of the film. As another example, the slitting of the edges can be planned and controlled according to the 2D map and, for example, broader areas near the lateral edges can be removed if the component distribution is uneven near the lateral edges.

The dry film may be considered as a thin continuous sheet formed material. Depending on its composition, purpose and properties, the dry film may also be considered as a thin paper or web, or even as a membrane.

In practical embodiments, a large number of data points may be provided, to thus allow for a detailed profile of the component content of the film to be derived. The measuring step may be performed after at least part of said at least one drying step.

Hence, the measuring may be performed after one or more drying steps, such as after the entire drying process, or measuring may be performed in between drying steps.

The method may further comprise at least one dewatering step prior to said at least one drying step, wherein said measuring step is performed after at least part of the at least one dewatering step and before said at least one drying step.

Hence, the measuring may be performed after one or more dewatering steps, such as after the entire dewatering process, but before a drying step, or in between sub-steps in the dewatering process.

Measurement may also be performed after release of the dry film from the support. Then measurement may be performed on the top side and/or back side of the film.

The method may further comprise a pre-drying step, which is performed prior to the at least one dewatering step and wherein said measuring step is performed after the pre-drying step and before the at least one dewatering step.

Said adjusting at least one production parameter may comprise varying a drying condition and/or varying a dewatering condition.

Varying a drying condition, and thus influencing the drying, implies that the moisture content is influenced and thereby the concentration of components that are soluble in the dispersing medium. The drying condition may be varied across all or part of a width of the MFC film on the support.

In a dewatering section, it is possible to vary a press nip and/or an alignment and/or a relative angle between press rolls.

It is also possible to change a press felt, such as to provide a press felt having different properties and/or to replace a press felt which is contaminated. Said varying a drying condition may comprise reducing the drying effect at at least one lateral edge portion of the wet MFC film.

The lateral edge portion may be defined as an area which extends in a direction perpendicular to a longitudinal direction (which is parallel with a length direction, MD) of the MFC film by a distance of 50 mm, preferably 30 mm or 10 mm, from an outermost edge (lateral edge) of the MFC film.

Said varying a drying condition may comprise guiding drying gas away from at least one lateral edge portion of the MFC film.

Said varying a drying condition may comprise extracting drying gas away from at least one lateral edge portion of the MFC film.

Said varying a drying condition may comprise masking, e.g. from drying gas and/or radiation, a lateral edge portion of the MFC film.

Said varying a drying condition may comprise sealing against a support surface laterally outside a lateral edge portion of the MFC film.

Hence the heating of the support can be reduced, leading to a reduction in the drying effect at edge portions of the film (F, F′).

Another example of varying the drying condition is controlling the temperature profile of the support, which may be heated or cooled from below.

Said varying a drying condition may comprise selectively controlling a duty cycle, a combustion gas pressure and/or an intensity of a radiation source for providing said drying.

Said varying a drying condition may comprise selectively injecting at least one of a cooling medium and a dispersing medium onto the wet MFC film.

Water may be used as cooling and/or dispersing medium.

Said varying a drying condition may comprise cooling at least one support edge portion laterally outside the wet MFC film by applying the cooling medium.

Said varying a drying condition may comprise applying said cooling medium on a lateral edge portion of the MFC film and/or applying the dispersing medium on a lateral edge portion of the MFC film.

The method may further comprise separating the dry MFC film from the support and winding the separated MFC film onto a reel.

Hence, the MFC film will remain on the support throughout the entire drying (and dewatering, if any) process.

Said adjusting at least one production parameter may comprise adjusting a dosing of said at least one component.

The dosing may be adjusted by reducing or increasing supply of one or more components. It is also conceivable to supply a further component in order to compensate for another component.

Said dosing may be adjusted upstream of said applying a layer of the MFC dispersion to the support, such as in an MFC supply.

Said dosing may be adjusted in a device for applying said layer of the MFC dispersion to the support.

Said adjusting at least one production parameter may comprise adjusting a mixing condition for the MFC dispersion.

The production parameter may be a mixing speed, a mixing time and/or a mixer duty cycle. It is also possible to engage one or more individually drivable mixing devices, as needed.

The production parameter may also be related to the supply of the component in that the position at which the component is supplied can be varied, either along a flow direction towards a casting device for applying a layer of the MFC dispersion to a support, or across the width of the casting device.

It is further possible to selectively supply a component to the wet MFC film surface once the MFC dispersion has been applied to the support.

The production parameter may also be a temperature, e.g. in an MFC supply, in a casting device and/or in a drying device.

The production parameter may be a delay time between e.g. an MFC supply and a casting device.

The production parameter may be a level (i.e. amount of MFC dispersion that is present) in e.g. the MFC supply and/or in a casting device.

It is also possible to adjust the supply of the MFC itself and/or of the dispersing medium.

Said mixing condition may be adjusted upstream of said applying a layer of the MFC dispersion to a support, such as in an MFC supply.

Said mixing condition may be adjusted in a device for applying said layer of the MFC dispersion to the support.

The provided MFC dispersion may have a dry solids content of about 2.5-25% by weight, preferably about 2.5-15% by weight, about 2.5-10% by weight or about 2.5-8% by weight.

Regardless of the dry solids content, a viscosity of the MFC dispersion may be above about 4 Pas at a shear rate of 20 s⁻¹.

The viscosity may be determined for a dispersion at a temperature of about 20-80 deg C. and preferably about 20-60 deg C. A preferred method of measuring viscosity is by use of a rheometer using bob-and-cup geometry, such as an Anton Paar MCR 302 dynamic rotational rheometer.

An average film thickness of the dry MFC film may be about 5-60 μm, preferably 10-50 μm, 15-45 μm or 20-40 μm.

A film grammage of the dry MFC film may be about 4-80 g/m², preferably 8-67 g/m², 12-60 g/m², 16-53 g/m2 or 20-45 g/m².

A dispersing medium content of the dry MFC film (F′) may be about 0.1-20% by weight, preferably 1-15% by weight, or 2-14% by weight.

A film forming component content of the dry MFC film (F′) may be at least 80-99.9% by weight, preferably 85-99% by weight, or 86-98% by weight.

The film forming component may comprise at least 60% by weight MFC, at least 70% by weight MFC or at least 80% by weight MFC.

A film width of the dry MFC film (F′) may be about 0.3-4 m, preferably 0.5-4 m, 1-4 m or 2-4 m.

The method may further comprise measuring said concentration of at least one component in the wet MFC film and/or in the dry MFC film at at least two data points which are spaced from each other in a thickness direction of the MFC film.

Measurement data indicating the component, such as additive or impurity, content at various points which are spaced apart in the thickness direction can be stored and used for following up MFC film quality and/or for grading the resulting MFC film.

Said component may be at least one unintended substance in said wet or dry MFC film.

Hence, the measurement may be used to detect contamination of the MFC film. Additionally, it is possible to detect the type of contamination, e.g. grease, oil, etc., which may facilitate location of the source of the contamination.

According to a second aspect, there is provided a device for producing a microfibrillated cellulose, MFC, film from an MFC dispersion. The device comprises a support guiding device, configured to guide a continuous support, a casting device, configured to apply the MFC dispersion as a wet MFC film on the support, a drying arrangement, configured for removing a dispersing medium from the wet MFC film so as to form a dry MFC film, at least one measuring arrangement, configured for providing data indicative of a concentration of at least one component in the wet MFC film and/or in the dry MFC film at at least two data points which are laterally spaced across a width of the MFC film, and/or which are longitudinally spaced along a length direction of the MFC film, and a controller, configured to receive said data, wherein the controller is configured to perform at least one of the tasks:
i) controlling at least one production parameter which influences the concentration of said at least one component in the MFC film; and
ii) recording said data as a chemical map of the dry MFC film, wherein a plurality of data points are each associated with a respective position on the dry MFC film.

The device may be configured for carrying out the method or methods as disclosed above.

The support guide may be configured to guide a support having a width of at least about 0.3-4 m, preferably 0.5-4 m, 1-4 m or 2-4 m.

The support may be a non-porous support and in particular a continuous non-porous support, such as a metal belt, in particular a steel belt, a polymer belt or a polymer coated belt. A metal belt may be coated, e.g., with ceramic material.

The device may further comprise a dewatering arrangement upstream said drying arrangement, wherein said measuring arrangement is arranged downstream of at least part of the dewatering arrangement.

Hence, the measuring may be performed downstream of one or more dewatering steps, such as downstream of the entire dewatering step, but before a drying step, or in between sub-steps in the dewatering process.

It is understood that the measuring arrangement may comprise a first measuring arrangement arranged at least partially downstream of the drying arrangement and a second measuring arrangement arranged at least partially downstream of the dewatering arrangement.

The device may further comprise a pre-drying arrangement, upstream said dewatering arrangement, wherein said measuring arrangement may be arranged downstream of at least part of the pre-drying arrangement and upstream of the dewatering arrangement.

A further measuring arrangement, or part of the measuring arrangement, may thus be arranged downstream of at least part of the pre-drying arrangement. According to a third aspect, there is provided use of a device as described above for forming a dry MFC film having a dispersing medium content of about 0.1-20% by weight, preferably 1-15% by weight, or 2-14% by weight, from an MFC dispersion having a dry solids content of about 2.5-25% by weight, preferably about 2.5-15% by weight, about 2.5-10% by weight or about 2.5-8% by weight, and optionally a viscosity which is above about 4 Pas at a shear rate of 20 s⁻¹.

According to a third aspect, there is provided a microfibrillated cellulose, MFC, film having a longitudinal direction, which is parallel with a length direction of the film and a width direction, which is perpendicular to the longitudinal direction, the MFC film having a film forming component content of at least 80-99.9% by weight, preferably 85-99% by weight, or 86-98% by weight, said film forming component comprising about 50-100% by weight MFC, the MFC film having a width of about 0.3-4 m, preferably 0.5-4 m, 1-4 m or 2-4 m, the MFC film having a dispersing medium content of about 0.1-20% by weight, preferably 1-15% by weight, or 2-14% by weight, from an MFC dispersion having a dry solids content of about 2.5-25% by weight, preferably about 2.5-15% by weight, about 2.5-10% by weight or about 2.5-8% by weight, the MFC film comprising an effective amount of at least one component, such as an additive or an impurity, and a component content of the MFC film having a standard deviation which is less than 1% by weight along the width direction of the MFC film, and/or along a length direction of the MFC film where the component content is analyzed for each cm of MFC film width.

In the MFC film, the film forming component may comprise at least 60% by weight MFC, at least 70% by weight MFC or at least 80% by weight MFC.

An average film thickness may be about 5-60 μm, preferably about 10-50 μm, about 15-45 μm or about 20-40 μm.

A film grammage may be about 4-80 g/m², preferably about 8-67 g/m², about 12-60 g/m², about 16-53 g/m² or about 20-45 g/m².

According to a fourth aspect, there is provided a method of producing a microfibrillated cellulose, MFC, film from an MFC dispersion. The method comprises providing an MFC dispersion, applying a layer of the MFC dispersion to a support to form a wet MFC film, subjecting the wet MFC film on the support to at least one drying step to form a dry MFC film, using a spectroscopic measuring arrangement to provide data indicative of a material composition of the wet MFC film and/or of the dry MFC film at at least two data points which are laterally spaced across a width of the MFC film, and/or which are longitudinally spaced along a length direction of the MFC film, and identifying at least one non-intended contaminant material based on said material composition data.

The MFC dispersion may have a dry solids content of about 2.5-25% by weight dispersed in at least one dispersing medium.

According to a fifth aspect, there is provided a device for producing a microfibrillated cellulose, MFC, film from an MFC dispersion. The device comprises a support guiding device, configured to guide a continuous support, a casting device, configured to apply the MFC dispersion as a wet MFC film on the support, a drying arrangement, configured for removing a dispersing medium from the wet MFC film so as to form a dry MFC film, at least one spectroscopic measuring arrangement, configured for providing data indicative of a material composition of the wet MFC film and/or of the dry MFC film at at least two data points which are laterally spaced across a width of the MFC film, and/or which are longitudinally spaced along a length direction of the MFC film, and a controller, configured to receive said data and to identify at least one non-intended contaminant material based on said material composition data indicative of a material composition.

### DRAWINGS

FIGS. 1a-1b schematically illustrate a film forming device.

FIG. 2 is a schematic sectional view taken along the line A-A in FIG. 1a, illustrating a first embodiment of a drying arrangement.

FIGS. 3a-3c are schematic sectional views taken along the line A-A in FIG. 1a, illustrating different versions of a second embodiment of a drying arrangement.

FIG. 4 is a schematic sectional view taken along the line A-A in FIG. 1a, illustrating a third embodiment of a drying arrangement.

FIG. 5 is a schematic sectional view taken along the line A-A in FIG. 1a, illustrating a fourth embodiment of a drying arrangement.

FIG. 6 is a schematic sectional view taken along the line A-A in FIG. 1a, illustrating a fifth embodiment of a drying arrangement.

FIG. 7 is a schematic sectional view taken along the line A-A in FIG. 1a, illustrating a sixth embodiment of a drying arrangement.

FIG. 8 is a schematic sectional view taken along the line A-A in FIG. 1a, illustrating a seventh embodiment of a drying arrangement.

FIG. 9 is a schematic sectional view taken along the line B-B in FIG. 1a, illustrating a first embodiment of a measuring arrangement.

FIG. 10 is a schematic sectional view taken along the line B-B in FIG. 1a, illustrating a second embodiment of a measuring arrangement.

FIG. 11 is a schematic sectional view taken along the line B-B in FIG. 1a, illustrating a third embodiment of a measuring arrangement.

FIG. 12 is a diagram illustrating a process for forming an MFC film.

### DETAILED DESCRIPTION

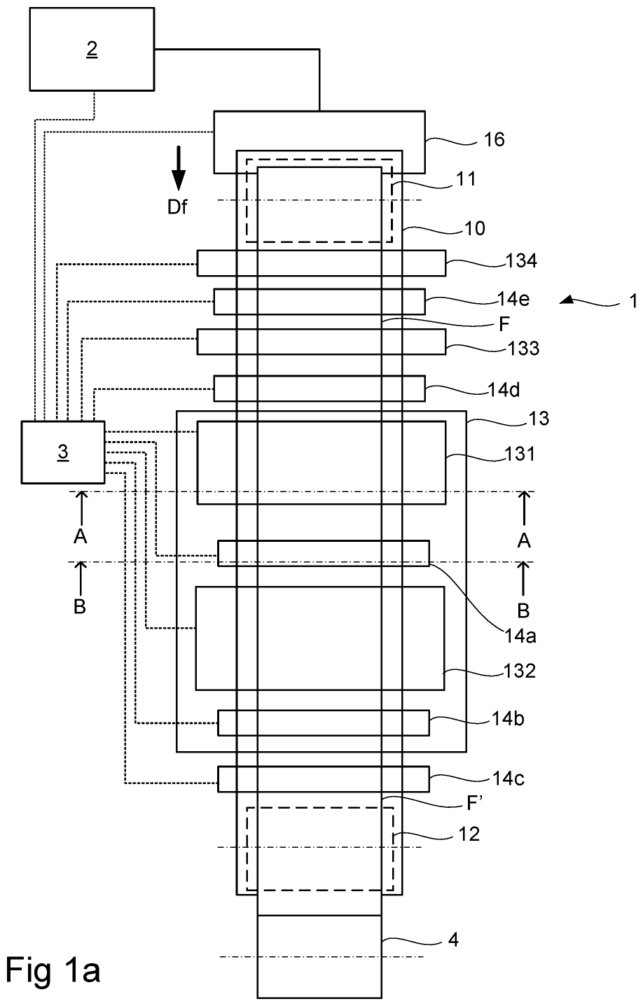

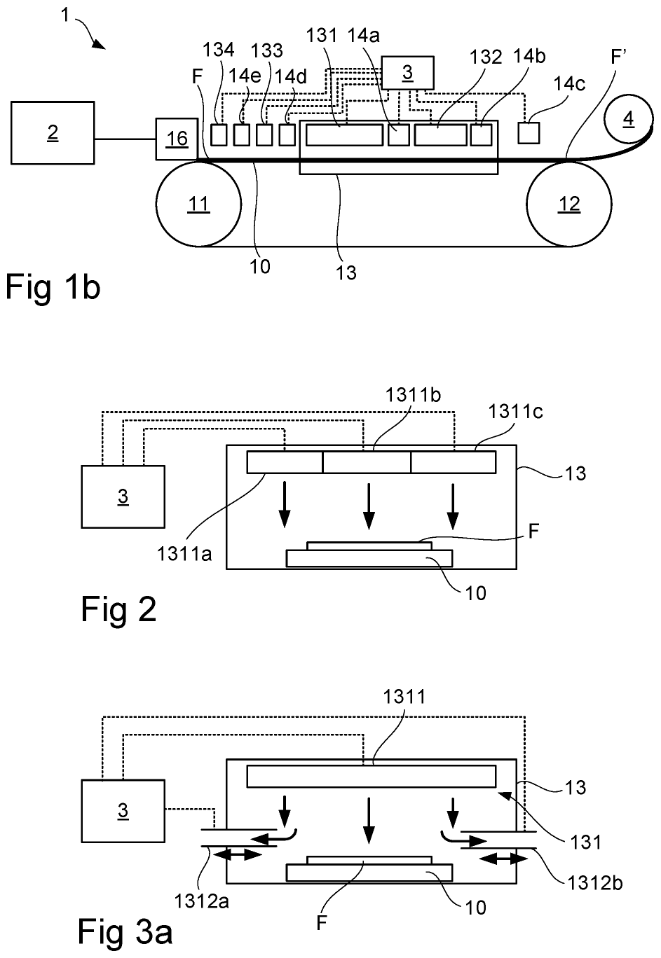

Referring to FIG. 1a, there is schematically illustrated a top view of a film forming device 1. FIG. 1b schematically illustrates a side view of the film forming device 1.

In the present disclosure, the film forming device 1 will be illustrated with reference to a film forming device 1 for forming a non-laminated film, or “free-standing film”, i.e. a film that is not laminated to any substrate material. Hence, as illustrated in FIGS. 1a-1b, the finished film F′ is stripped off a support 10 and rolled onto a reel 4.

The support 10 from which the dry film F′ is to be stripped off may be provided in the form of a non-porous and preferably endless belt, such as a metal belt, e.g. a steel belt or a polymer, or polymer coated, belt. A metal belt may be coated by e.g. a ceramic coating.

The support 10 is preferably non-porous, so as to provide for a smooth film surface. In particular, the support may be polished to mirror gloss. In the illustrated embodiment, the support 10 is an endless support which runs over a support guide in the form of a pair of pulleys 11, 12.

An MFC supply 2 is provided for supplying an MFC dispersion to a casting device 16, which is configured to deposit the MFC dispersion as a thin, wet film F having an even thickness. The support 10 carrying the wet film F is passed through a dryer 13, which may comprise one or more drying arrangements 131, 132. In the case with more than one drying arrangement 131, 132, the drying arrangements may be identical with each other, or they may differ in terms of e.g. length. Also, the drying arrangements 131, 132 may be individually controlled, such that they provide different drying parameters.

The MFC supply 2 may comprise one or more mixing arrangements (not shown) for mixing the MFC, the dispersing medium and additives, if any.

The mixing arrangement can be suitable for mixing viscous material, meaning, for example, a ribbon agitator with double helical design. In such design, the outer helix moves the material in one direction and inner helix into the opposite direction, which mixes the whole volume efficiently. In certain cases, there is a need for high shear forces and high shear mixer, like a saw-tooth disperser blade or rotor-stator disperser. The high shear mixer can be used alone or together with another mixer. In addition to, or as an alternative, the mixing arrangement, there can be pipeline equipped with a pump which moves material from the bottom of the MFC supply to top and in that way improves the distribution of the additives in the vertical direction. Uneven distribution of the additives in the wet film For dry film F′ can be related to insufficient mixing in the MFC supply 2 which can be improved by higher mixing speed and higher shear rate.

The mixing arrangements may be controllable in terms of operating speed, temperature and/or duty cycle. Control of the mixing arrangements may be provided by a separate controller or by controller 3, which controls also other parts of the film forming device 1.

The MFC supply 2 may also comprise a controllable arrangement for selectively supplying one or more additives to the MFC dispersion.

In the MFC supply 2 it may be possible to control temperature throughout, or in parts of, the MFC supply.

The MFC supply 2 may also comprise a controllable heating/cooling arrangement which can control the temperature of the MFC dispersion. Sometimes, increasing the temperature improves the distribution of the additives since it decreases the viscosity of the MFC dispersion and makes the mixing more efficient.

On the other hand, too high temperature can lead to thermal degradation of certain additives and thus bring unwanted substances to the film. The temperature can be controlled, for example, with heat exchanger, heating/cooling jacket around the chest and/or with direct or indirect steam.

Moreover, the MFC supply 2 can comprise pH and/or conductivity adjustment arrangement. For example, this arrangement can consist of a possibility to add acid and/or base to the MFC dispersion and measurement of pH in the MFC dispersion. In another example, this arrangement can consist of a possibility to add salt solution to the MFC dispersion and measure the conductivity of the MFC suspension. pH and/or conductivity of the dispersion can affect the surface charge of all or some components which in turn affects how well they mix with each other. Sometimes, changes in pH can lead to chemical reactions between components which is visible in the chemical composition measured from the wet or dry film.

The casting device 16 may comprise one or more mixing arrangements (not shown) for mixing the MFC, the dispersing medium and additives, if any. The mixing arrangements may be controllable in terms of operating speed and/or duty cycle.

The casting device 16 may also comprise a controllable arrangement for selectively supplying one or more additives to the MFC dispersion. Also in the casting device, it may be possible to control temperature in parts of, or throughout, the casting device 16.

Optionally, a dewatering arrangement 133, such as a dewatering device, which may include a press, may be provided upstream of the drying arrangements 131, 132. Such dewatering arrangements 133 are known as such.

For example, the dewatering may be performed by applying a press fabric in direct contact with the wet MFC film and conducting the wet MFC film, arranged between the press fabric and the support, through a pressing equipment. Alternatively, the dewatering may be performed by applying a porous wire or membrane in direct contact with the wet MFC film and conducting the wet MFC film, arranged between the porous wire or membrane and the support, through a vacuum dewatering equipment, in which the porous wire or membrane is covering one or several vacuum cavities that causes dispersing medium to be removed from the wet MFC film.

Further, optionally, a pre-drying arrangement 134 may be provided upstream of the dewatering arrangement 133.

In other embodiments, the drying arrangements 131, 132, and pre-drying arrangement 134, if any, may use the same or different drying techniques, e.g. each one being selected according to the non-limiting options mentioned herein.

One or more measuring arrangements 14a, 14b, 14c, 14d, 14e for measuring at least one parameter indicative of a concentration of at least one component, such as an additive or a chemical composition in the wet MFC film F and/or in the dry MFC film F′ are provided, either inside the dryer 13 or outside the dryer 13. For example, a measuring arrangement 14a may be provided inside the dryer 13, between a pair of drying arrangements 131, 132. As another example, a measuring arrangement 14b may be provided inside the dryer 13 and downstream of the drying arrangements 131, 132. As yet another example, a measuring arrangement 14c may be provided downstream of and outside the dryer 13. For example, a cooling arrangement (not shown) may be provided between the dryer 13 and the measuring arrangement 14c.

It is understood that one, two, three, four or all of the above illustrated measuring arrangements 14a, 14b, 14c, 14d, 14e may be provided.

Where a dewatering arrangement 133 is provided upstream of the drying arrangement 131, 132, a measuring device 14d may be provided downstream of the dewatering arrangement 133 and upstream of the drying arrangement 131, 132. In some embodiments, the measuring arrangement, or a sub-part thereof, may be provided downstream of part of the dewatering arrangement 133, such as between sub-steps of the dewatering arrangement 133, or between dewatering arrangements, if several dewatering arrangements are provided.

It is also possible to provide a measuring arrangement 14e upstream of the dewatering arrangement 133.

A controller 3 may be provided for controlling at least the dryer 13 and the measuring arrangements 14a, 14b, 14c, 14d, 14e. Optionally, the controller may control further, or all, functions of the film forming device 1. The controller 3 may also control the dewatering arrangement 133, if any, as well as the pre-drying arrangement 134, if any.

The controller 3 may also control the operation of the MFC supply 2 and/or of the casting device 16. In particular, control of the mixing arrangements, of the arrangement for supplying one or more additives, of temperature, pH and/or conductivity may be provided by a separate controller or by the controller 3, which controls also other parts of the film forming device 1.

FIG. 2 is a schematic sectional view taken along the line A-A in FIG. 1a, illustrating a first embodiment of a drying arrangement, which may be arranged in a dryer 13.

Drying and predrying may be performed by evaporation, impingement drying with hot gas/air, infra-red (IR) drying, microwaves, near infrared (NIR) drying, UV drying, radiation drying, thermal heating, heating the support with steam or electricity or any other method or combination of methods well known in the art.

It is understood that any of the herein mentioned drying techniques can be made controllable across the width and/or along the length of the support 10. For example, also techniques that heat the support 10 from below can easily be divided into zones, as desired.

The drying arrangement illustrated in FIG. 2 is a controllable convection drying arrangement, for selectively feeding a dry and preferably also warm or hot gas, such as air, towards the wet film F.

In the drying arrangement illustrated in FIG. 2, three individually controllable convection zones 1311a, 1311b, 1311c are provided. It is understood that the number of convention zones 1311a, 1311b, 1311c can be selected depending on the width of the film and support and on what resolution is required.

For each convection zone 1311a, 1311b, 1311c, a gas flow rate and/or a gas flow direction and/or a gas temperature and/or a gas composition (e.g. moisture content) may be individually controllable by the controller 3, either by controlling a respective blower, a respective nozzle, a respective heater and/or a respective gas mixer. Alternatively, or as a supplement, the supply of the gas to the respective convection zone 1311a, 1311b, 1311c may be controlled by a valve (not shown).

Hence, during operation, the drying effect in each of the zones 1311a, 1311b, 1311c is individually controlled by the controller 3. For example, it is possible to provide a lower gas flow and/or lower temperature and/or higher moisture content at the zones 1311a, 1311c near the lateral edges of the film F, so as to reduce the drying effect at the lateral edge portions of the film F.

In the embodiment of FIG. 2, it is possible to provide for a drying gas impingement width to be narrower than that of the MFC film F, F′. For example, the impingement width may be 30-70 mm, preferably 40-60 mm or about 50 mm, narrower than a film F, F′ width.

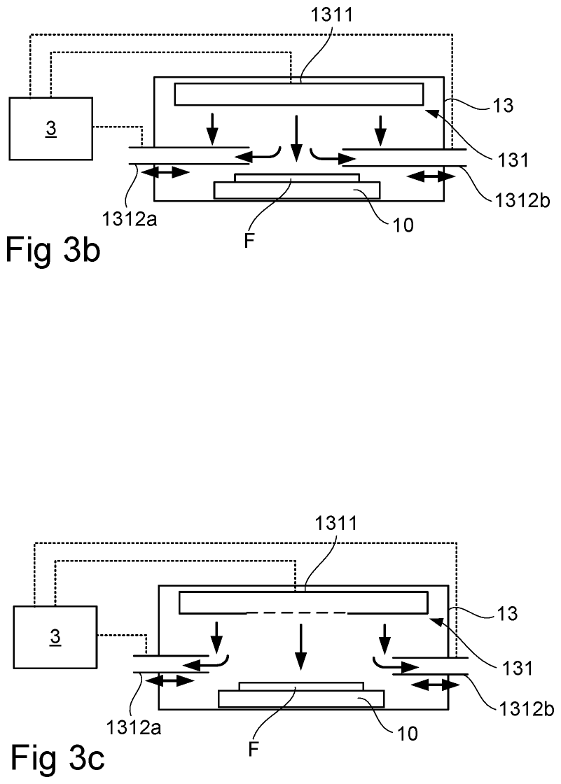

FIG. 3a is a schematic sectional view taken along the line A-A in FIG. 1a, illustrating a first version of a second embodiment of a drying arrangement, which may be arranged in a dryer 13.

In the drying arrangement illustrated in FIG. 3a, there is a single convection zone 1311, which may be operated in the same manner as one of the convection zones described with reference to FIG. 2, in that a gas flow rate and/or a gas flow direction and/or a gas temperature and/or a gas composition (e.g. moisture content) may be controllable by the controller 3.

In FIG. 3a, there is provided at least one evacuation outlet 1312a, 1312b, which may be laterally and/or vertically displaceable, optionally controllable by the controller 3, such that its outlets can be positioned at a desired lateral position relative to the film F. The evacuation outlets 1312a, 1312b may be connected to an extraction device, such as a fan, which may be controllable by the controller 3, such that an extraction rate is controllable by the controller 3.

FIG. 3b schematically illustrates a second version of the second embodiment of a drying arrangement, wherein inlets to the evacuation outlets 1312a, 1312b are positioned laterally inwardly of MFC film F, F′ edges, such that the drying gas is prevented from reaching the MFC film edges.

FIG. 3c schematically illustrates a third version of the second embodiment of a drying arrangement, wherein the convection zone 1311 is configured to provide a greater flow of drying gas at a laterally central portion thereof, e.g. by providing reduced gas flow resistance at the central portion of the convection zone 1311 as compared with at edge portions of the convection zone 1311. This embodiment may be combined with the embodiments of FIGS. 3a and/or 3b.

Hence, during operation, the drying effect at lateral edge portions of the film F may be reduced by dry and/or hot air being extracted from the area at the lateral edge portions and thus being prevented from interacting with the wet film F, so as to reduce the drying effect at the lateral edge portions of the film F.

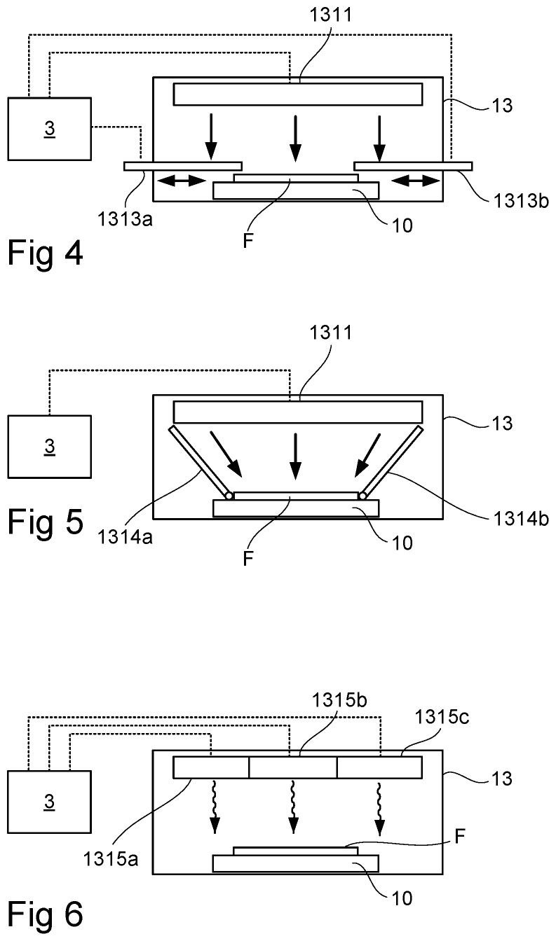

FIG. 4 is a schematic sectional view taken along the line A-A in FIG. 1a, illustrating a third embodiment of a drying arrangement, which may be arranged in a dryer 13.

In the drying arrangement illustrated in FIG. 4, there is a single convection zone 1311, which may be operated in the same manner as the convection zone described with reference to FIGS. 3a-3c.

In FIG. 4, there is provided at least one shield 1313a, 1313b, which may be laterally displaceable, optionally controllable by the controller 3, such that the shields 1313a, 1313b can be positioned at a desired lateral position relative to the film F.

Hence, during operation, the drying effect at lateral edge portions of the film may be reduced as the incoming hot and/or dry gas is deflected from the lateral edge portions, so as to reduce the drying effect at the lateral edge portions of the film F.

FIG. 5 is a schematic sectional view taken along the line A-A in FIG. 1a, illustrating a fourth embodiment of a drying arrangement, which may be arranged in a dryer 13.

In the drying arrangement illustrated in FIG. 5, there is a single convection zone 1311, which may be operated in the same manner as the convection zone described with reference to FIGS. 3a-3c.

In FIG. 5, there is provided at least one sealing arrangement 1314a, 1314b, which may seal against the support 10 immediately laterally outside the film F, such that the gas flow from the convection zone 1311 does not impinge on the support 10.

Hence, during operation, the hot gas is prevented from reaching the support 10, which may be a metal support and as such having higher heat coefficient than the film F, whereby the heating of the support laterally outside of the film F is reduced, and thus also the drying effect at the lateral edge portion of the film F.

FIG. 6 is a schematic sectional view taken along the line A-A in FIG. 1a, illustrating a fifth embodiment of a drying arrangement, which may be arranged in a dryer 13.

The drying arrangement illustrated in FIG. 6 is a controllable radiation drying arrangement, for selectively projecting radiation, such as infra-red (IR) radiation towards the wet film F.

In the drying arrangement illustrated in FIG. 6, three individually controllable radiation zones 1315a, 1315b, 1315c are provided. It is understood that the number of radiation zones 1315a, 1315b, 1315c can be selected depending on the width of the film F and the support 10 and on what resolution is required.

For each radiation zone 1315a, 1315b, 1315c, a radiation intensity and/or a radiation duty cycle may be individually controllable, by the controller 3, e.g. by controlling the respective radiation source and/or by controlling a radiation filter or valve.

Hence, during operation, the drying effect in each of the radiation zones 1315a, 1315b, 1315c is individually controlled by the controller 3. For example, it is possible to provide radiation in the radiation zones 1315a, 1315c near the film lateral edges, so as to reduce the drying effect at the lateral edge portions of the film F.

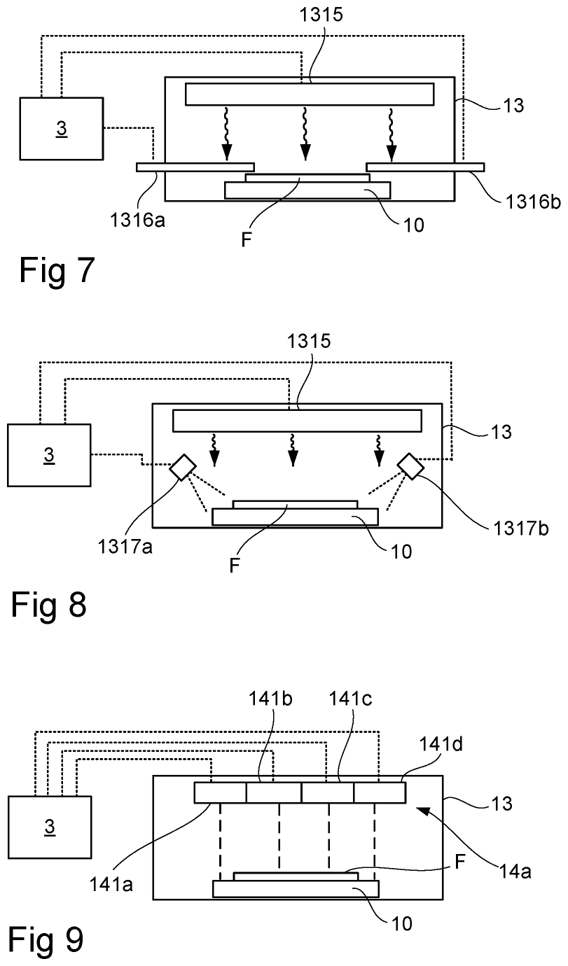

FIG. 7 is a schematic sectional view taken along the line A-A in FIG. 1a, illustrating a sixth embodiment of a drying arrangement, which may be arranged in a dryer 13.

In the drying arrangement illustrated in FIG. 7, there is a single radiation zone 1315, which may be operated in the same manner as one of the radiation zones described with reference to FIG. 6.

In FIG. 7, there is provided at least one shield 1316a, 1316b, which may be laterally displaceable, optionally controllable by the controller 3, such that the shields 1316a, 1316b can be positioned at a desired lateral position relative to the film F. The shields may be completely non-transparent to the radiation. Alternatively, the shields may be variably, e.g. controllable by the controller 3, transparent to the radiation or partially transparent to the radiation.

The shields 1316a, 1316b may be operated so as to mask only the support 10 so as to reduce heating of the support 10, or so as to mask both the support 10 and lateral edge portions of the film F.

Hence, during operation, the drying effect at lateral edge portions of the film may be reduced as the incoming radiation is masked from the support 10 and optionally also from the lateral edge portions, so as to reduce the drying effect at the lateral edge portions of the film F.

FIG. 8 is a schematic sectional view taken along the line A-A in FIG. 1a, illustrating a seventh embodiment of a drying arrangement, which may be arranged in a dryer 13.

In the drying arrangement illustrated in FIG. 8, there is a single radiation zone 1315, which may be operated in the same manner as one of the radiation zones described with reference to FIG. 6.

In FIG. 8, there is provided at least one injector 1317a, 1317b for a dispersing medium and/or a coolant agent. The injector 1317a, 1317b may be controllable by the controller 3 so as to selectively apply dispersing medium and/or coolant agent to the film F and/or to the support 10 just laterally outside the film, so as to increase the moisture level in the film, and/or to cool the film F and/or the support 10.

Hence, during operation, the drying effect at lateral edge portions of the film may be reduced as the moisture level of the film F is selectively increased and/or as the support 10 and optionally also the lateral edge portions are cooled, so as to reduce the drying effect at the lateral edge portions of the film F. Hence, by adjusting the drying effect, and dewatering effect, if any, the migration of additives may also be adjusted.

FIG. 9 is a schematic sectional view taken along the line B-B in FIG. 1a, illustrating a first embodiment of a measuring arrangement, which may be arranged inside or outside a dryer 13.

In the measuring arrangement 14a illustrated in FIG. 9, a measuring sensor is connected to the controller 3 and may be formed as a 1D sensor (a line sensor) having a plurality of sensor zones 141a, 141b, 141c, 141d, each capable of generating sensor data for a laterally limited portion of the film F and optionally of the support 10. The number of sensor zones 141a, 141b, 141c, 141d may be arbitrarily selected depending on the required resolution.

Various sensing techniques may be utilized.

For example, the sensor may use infra-red (IR) spectroscopy, near infra-red (NIR) spectroscopy or Raman spectroscopy to provide data corresponding to a composition of the film F, whereby a material composition may be derived based on resulting spectral data.

It is possible to use spectroscopy methods, such as near infra-red spectroscopy (NIR), Raman spectroscopy, to not only measure the dry solids content and/or chemical composition at a point on the surface of the wet or dry MFC film, but also to measure the dry solids content and/or chemical composition at various points along a thickness direction of the wet or dry MFC film F, F′.

Hence, effectively, a 2D map or a 3D map of the additive content of the wet or dry MFC film F, F′ may be created.

The sensor may be operated continuously or at certain intervals to derive a composition profile of the film F, which may be used as input to the controller 3 for determining how to operate the drying arrangement, the casting device 16 and/or the MFC supply 2.

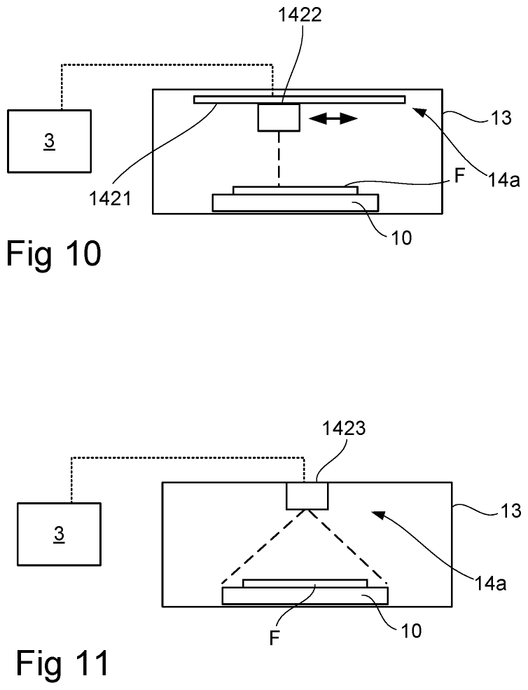

FIG. 10 is a schematic sectional view taken along the line B-B in FIG. 1a, illustrating a second embodiment of a measuring arrangement, which may be arranged inside or outside a dryer 13.

In the measuring arrangement illustrated in FIG. 10, a measuring sensor 1422 is connected to the controller 3 and may be formed as a point sensor, which may be scanned across the film F, F′ and optionally also across the support 10. Scanning may be achieved by displacing the measuring sensor along a guide 1421 and/or by using a beam guide.

The sensor may use any of the sensing techniques described with reference to FIG. 9.

As with the arrangement described with reference to FIG. 9, the sensor 1422 may be operated continuously or at certain intervals to derive a temperature or composition profile of the film F, F′, which may be used as input to the controller 3 for determining how to operate the drying arrangement 13, the casting device 16 and/or the MFC supply 2.

FIG. 11 is a schematic sectional view taken along the line B-B in FIG. 1a, illustrating a third embodiment of a measuring arrangement, which may be arranged inside or outside a dryer 13.

In the measuring arrangement illustrated in FIG. 11, a 2D sensor, such as a camera 1423, e.g. a hyperspectral camera or an infra-red (IR) camera, may be arranged such that a field of view covers the width of the film F and optionally also of the support 10.

The camera 1423 may thus use any of the sensing techniques described with reference to FIG. 9.

As with the arrangement described with reference to FIG. 9, the camera 1423 may be operated continuously or at certain intervals to derive a composition profile of the film F, which may be used as input to the controller 3 for determining how to operate the drying arrangement 13, the casting device 16 and/or the MFC supply 2.

At least one measuring arrangement as disclosed with respect to any one of FIGS. 9-11 may be applied at any position along the support 10 where a measuring arrangement 14a, 14b, 14c, 14d, 14e is being indicated.

A wet MFC film F can be formed from an MFC dispersion having a dry solids content about 2.5-4% by weight, about 4-6% by weight, about 6-8% by weight, about 8-10% by weight, about 10-12% by weight, about 12-14% by weight, about 14-16% by weight, about 16-18% by weight, about 18-20% by weight, about 20-22% by weight or about 22-25% by weight, which is considered a high dry solids content MFC. Preferably, the dry solids content may be greater than 3% or greater than 4% by weight.

Thickness of the dry film F′ may be measured using, as non-limiting examples, white light interferometry, laser profilometry, or optically by cutting a sample in cross-machine directional line (either cast in resin or not) and microscopic imaging (e.g. scanning electron microscopy or other applicable method) of the cut section in thickness direction.

The average dry film F′ thickness may be on the order of 5-60 μm, 15-20 μm, preferably 20-60 μm, 10-50 μm, 30-50 μm, 15-45 μm or 20-40 μm.

Particular dry film F′ thicknesses may be 5-10 μm, 10-15 μm, 15-20 μm, 20-25 μm, 25-30 μm, 30-35 μm, 35-40 μm, 40-45 μm, 45-50 μm, 50-55 μm or 55-60 μm.

A dry film F′ grammage may be on the order of 4-80 g/m2, preferably 8-67 g/m², 12-60 g/m², 16-53 g/m² or 20-45 g/m².

Particular dry film F′ grammages may be 4-10 g/m², 10-20 g/m², 20-30 g/m², 30-40 g/m², 40-50 g/m², 50-60 g/m², 60-70 g/m² or 70-80 g/m².

A dispersing medium content of the dry film F′ may be on the order of 0.1-20% by weight, preferably 1-15% by weight, or 2-14% by weight.

Particular dispersing medium contents of the dry film F′ may be on the order of 0.1-1% by weight, 1-2% by weight, 2-3% by weight, 3-4% by weight, 4-5% by weight, 5-6% by weight, 6-7% by weight, 7-8% by weight, 8-9% by weight, 9-10% by weight, 10-11% by weight, 11-12% by weight, 12-13% by weight, 13-14% by weight or 14-15% by weight.

A film forming component content of the dry film F′ may be 80-99.9% by weight, preferably 85-99% by weight, or 86-98% by weight, with the remainder being dispersing medium and/or one or more additives.

In particular, the film forming component of the dry film F′ may have an MFC content of 50-60% by weight, 60-70% by weight, 70-80% by weight, 80-90% by weight, 90-95% by weight or 95-99% by weight, with the remainder being dispersing medium and/or one or more additives.

A width of the dry film F′ may be about 0.3-4 m, preferably 0.5-4 m, 1-4 m or 2-4 m. Particular film F, F′ widths may be 0.3-0.5 m, 0.5-1 m, 1-1.5 m, 1.5-2 m, 2-2.5 m, 2.5-3 m, 3-3.5 m or 3.5-4 m. A corresponding support 10 width may be at least the same as the film width and in some cases about 10-20 cm wider than the film width.

By using the method according to the present disclosure, it is possible to produce a dry MFC film F′ having an even composition with respect to the dispersing medium and/or additives, if any.

By measuring the content of the dispersing medium and/or chemical composition of the film at a plurality of points across the width of the dry MFC film F′, such as at 1 cm intervals across the entire width of the dry MFC film F′, it is possible to derive an average content of the dispersing medium and/or additives, as well as a standard deviation of said content. With the method disclosed herein, it is possible to achieve a standard deviation of 1% by weight or less, also across the width of an MFC film F′ having a width which exceeds that of lab scale equipment.

It is also possible to impact the dry solids content and additive distribution and content of the film F, F′ by measures which may be taken at the casting device 16.

For example, it may be possible to adjust a nozzle of the casting device 16 so as to vary a thickness of the wet MFC dispersion that is applied to the support 10, for example so as to increase thickness in portions where the MFC film F, F′ dries faster.

It is also possible to vary the temperature at the nozzle, such that the temperature of the wet MFC film F, F′ applied to the support 10 will vary across the width of the support 10.

It is also possible to locally cool or heat the support 10 at or immediately downstream of the casting device 16.

It is also possible to detect impurities or collections of components present on, such as stuck to, the support 10, indicating that cleaning of the non-porous support is needed. Impurities or collections of components present on the support 10 may imply that impurities are added to the film or that an uneven distribution of components/additives is provided.

It is also possible to detect a partial clogging or impurities in the nozzle which may cause uneven thickness of the film or additive distribution in the film.

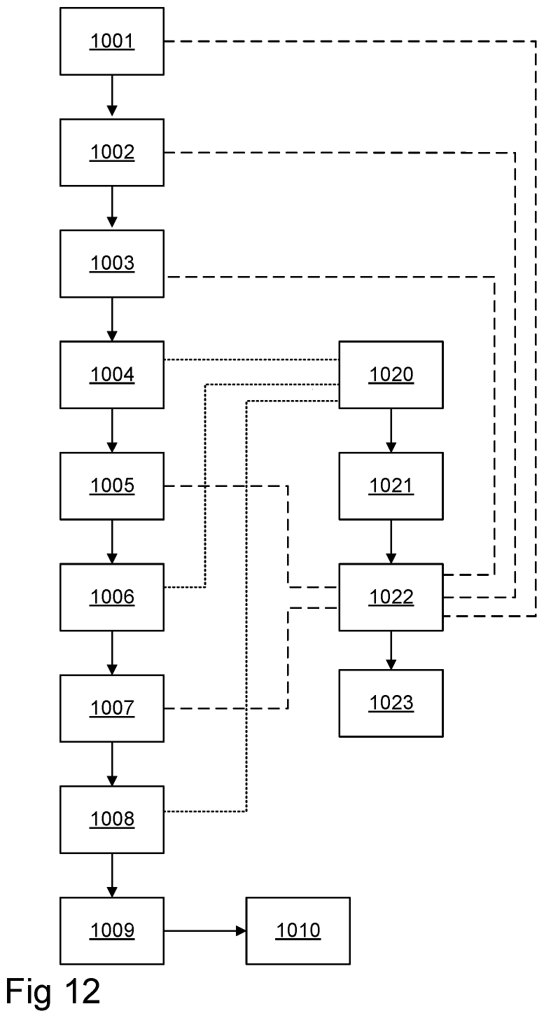

FIG. 12 schematically illustrates a method in accordance with the present disclosure.

Step 1001 represents a mixing operation, during which the MFC is mixed with dispersing medium and optionally additives to form the MFC dispersion.

Step 1002 represents an application operation, during which the MFC dispersion is applied to the support 10.

Step 1003 represents a pre-drying step, which is optional, and which may be performed in accordance with any of the drying methods disclosed above.

Step 1004 represents a first measuring step, which is optional, and which may be performed in accordance with any of the measuring methods disclosed above.

Step 1005 discloses a dewatering step, which is optional, and which may be performed in accordance with any of the dewatering methods disclosed above.

Step 1006 represents a second measuring step, which may be performed in accordance with any of the measuring methods disclosed above. The second measuring step is only used if preceded by a dewatering step 1005.

Step 1007 represents a main drying step, which may be performed in accordance with any of the drying methods disclosed above.

Step 1008 represents a third measuring step, which may be performed in accordance with any of the measuring methods disclosed above.

Step 1009 represents a stripping step, wherein the dry MFC film F′ is removed from the support 10, e.g. by a doctor blade.

Step 1010 represents a packaging step, wherein the dry MFC film F′ is packaged for shipping, e.g. by being rolled onto a reel.

Step 1020 represents a measurement data receiving step, wherein measurement data or signals from the measurement steps 1004, 1006, 1008 is/are received, e.g. in the controller 3.

Step 1021 represents a processing step, wherein the measurement data or signals is/are processed in order to derive information on the composition of the MFC film F, F′ at the respective measuring point.

Step 1022 represents a control step, wherein control data or control signals is/are sent as input for controlling one or more of the mixing step 1001, the application step 1002, the predrying step 1003, the dewatering step 1005 and the drying step 1007, in accordance with what has been disclosed above.

Step 1023 represents a mapping step, wherein a compositional map in 2D or 3D is created, representing the composition of the dry MFC film. The mapping step 1023 may be based on data received in one or more of the measuring steps 1004, 1006, 1008.

1. A method of producing a microfibrillated cellulose, MFC, film from an MFC dispersion, comprising:
providing an MFC dispersion comprising a dispersing medium and a film forming component comprising about 50-100% by weight MFC,
applying a layer of the MFC dispersion to a support to form a wet MFC film,
subjecting the wet MFC film on the support to at least one drying step to form a dry MFC film,
measuring at least one parameter indicative of a concentration of at least one component in the wet MFC film, in the dry MFC film, or both at at least two data points which are laterally spaced across a width of said MFC film, or which are longitudinally spaced along a length direction of said MFC film, or both, and
recording said at least one parameter for each of said data points to provide a chemical map of the dry MFC film, the chemical map comprising a plurality of data points which are each associated with a respective position on the dry MFC film.

2. The method as claimed in claim 1, wherein said measuring is performed after at least part of said at least one drying step.

3. The method as claimed in claim 1, further comprising:
at least one dewatering step prior to said at least one drying step, wherein said measuring is performed after at least part of the at least one dewatering step and before said at least one drying step.

4. The method as claimed in claim 3, further comprising:
a pre-drying step, which is performed prior to the at least one dewatering step and wherein said measuring is performed after the pre-drying step and before the at least one dewatering step.

5.-14. (canceled)

15. The method as claimed in claim 1, further comprising:
separating the dry MFC film from the support and winding the separated MFC film onto a reel.

16.-21. (canceled)

22. The method as claimed in claim 1, wherein the MFC dispersion has a dry solids content of about 2.5-25% by weight, and optionally a viscosity which is above about 4 Pas at a shear rate of 20 s⁻¹.

23. The method as claimed in claim 1, wherein an average film thickness of the dry MFC film is about 5-60 μm.

24. The method as claimed in claim 1, wherein a film weight of the dry MFC film is about 4-80 g/m².

25. The method as claimed in claim 1, wherein a dispersing medium content of the dry MFC film is about 0.1-20% by weight.

26. The method as claimed in claim 1, wherein a film forming component content of the dry MFC film is at least 80-99.9% by weight.

27. The method as claimed in claim 26, wherein the film forming component comprises at least 60% by weight MFC.

28. The method as claimed in claim 1, wherein a film width of the dry MFC film is about 0.3-4 m.

29. The method as claimed in claim 1, further comprising:
measuring said at least one parameter indicative of the concentration of at least one component in the wet MFC film, or in the dry MFC film, or both at at least two data points which are spaced from each other in a thickness direction of the MFC film.

30. The method as claimed in claim 1, wherein said component is at least one unintended substance in said wet MFC film or said dry MFC film.

31. A device for producing a microfibrillated cellulose, MFC, film from an MFC dispersion, comprising:
a support guiding device, configured to guide a continuous support,
a casting device, configured to apply an MFC dispersion as a wet MFC film on the support,
a drying arrangement, configured for removing a dispersing medium from the wet MFC film so as to form a dry MFC film,
at least one measuring arrangement, configured for providing data indicative of a concentration of at least one component in the wet MFC film or in the dry MFC film, or both at at least two data points which are laterally spaced across a width of the MFC film, or which are longitudinally spaced along a length direction of the MFC film, or both, and
a controller, configured to receive said data, wherein the controller is configured to:
record said data as a chemical map of the dry MFC film, wherein a plurality of data points are each associated with a respective position on the dry MFC film.

32.-36. (canceled)

37. A microfibrillated cellulose, MFC, film having a longitudinal direction, which is parallel with a length direction of the MFC film and a width direction, which is perpendicular to the longitudinal direction,
the MFC film having a film forming component content of at least 80-99.9% by weight,
said film forming component comprising about 50-100% by weight MFC,
the MFC film having a width of about 0.3-4 m, preferably 0.5-4 m,
the MFC film having a dispersing medium content of about 0.1-20% by weight,
the MFC film comprising at least one component, such as an additive or an impurity, and
a component content of the MFC film having a standard deviation which is less than 1% by weight along the width direction of the MFC film, or along a length direction of the MFC film, or both, where the component content is analyzed for each cm of MFC film width.

38.-42. (canceled)
